SEQUENCE LISTING (1 sequence)

SEQ ID NO: 1 - protein, 221 aa, synthetic construct
WAHVGGQDKPFCSAWPSAVVPQGGHVTLRCHYRRGFNIFTLYKKDGVPVPELYNRIFWNSFLISPVTPAHAGTYRCRGFHPHSPTEWSAPSNPLVIMVTGLYEKPSLTARPGPTVRAGENVTLSCSSQSSFDIYHLSREGEAHELRLPAVPSINGTFQADFPLGPATHGETYRCFGSFHGSPYEWSDPSDPLPVSVTGNPSSSWPSPTEPSFKTGIARHLH

(19) United States
(12) Patent Application Publication
(10) Pub. No.: US 2026/0159584 A1
(43) Pub. Date: Jun. 11, 2026

(54) CD158D MOLECULE, NEUTRALIZING ANTIBODY THEREOF, AND USE THEREOF

(71) Applicant: SUN YAT-SEN MEMORIAL HOSPITAL, Guangzhou, Guangdong (CN)

(72) Inventors: Erwei SONG, Guangzhou, Guangdong (CN); Jianing CHEN, Guangzhou, Guangdong (CN); Wenfeng ZENG, Guangzhou, Guangdong (CN)

(21) Appl. No.: 18/998,668

(22) PCT Filed: Jul. 25, 2023
(86) PCT No.: PCT/CN2023/109181

§ 371 Date: Jan. 27, 2025

(30) Foreign Application Priority Data
Jul. 26, 2022 (CN) 202210886386.5

(51) Int. Cl. C07K 16/28; A61K 39/00; A61P 35/00; C07K 14/705; G01N 33/50

(52) U.S. Cl. CPC C07K 16/2803; A61P 35/00; C07K 14/70503; G01N 33/5011; A61K 2039/505; C07K 2317/76; G01N 2333/70503; G01N 2333/91194

(57) ABSTRACT

A CD158d molecule, a neutralizing antibody thereof, and use thereof are provided. HS3ST3B1 positive tumor-related fibroblasts can mediate the death of killing lymphocytes and a new immune checkpoint molecule CD158d has been discovered. Based on the fact that the CD158d molecule can mediate the death of killing lymphocytes which is mediated by tumor fibroblasts, a neutralizing antibody capable of neutralizing the CD158d molecule has been obtained. Application of the CD158d molecule as a target in screening for or preparing a medicament for inhibiting tumor immune escape, and use of the CD158d neutralizing antibody in preparing a medicament for inhibiting tumor immune escape have been developed.

The present application claims priority to the prior application with the patent application No. 202210886386.5 and entitled “CD158D MOLECULE, NEUTRALIZING ANTIBODY THEREOF, AND USE THEREOF” filed with the China National Intellectual Property Administration on Jul. 26, 2022, which is incorporated herein by reference in its entirety.

### TECHNICAL FIELD

The present disclosure relates to a CD158d molecule, a neutralizing antibody thereof, and use thereof, and relates to the fields of bioengineering and biotherapy.

### BACKGROUND

Tumors are one of the major diseases threatening human life and health. The understanding and research of human beings on tumors have undergone a long process. The current treatment for tumors primarily involves a comprehensive strategy that encompasses surgery, chemotherapy, radiotherapy, and targeted therapy. In recent years, the survival status of tumor patients has significantly improved through the rational application of comprehensive treatment strategies involving these therapeutic approaches. However, distant metastasis, recurrence and treatment tolerability of tumors remain the final challenges for many tumor patients and are the dilemma of the current malignant tumor treatment.

The immune system is an important defense barrier of the body. It identifies and eliminates “non-self” components that appear within the body and maintains internal environmental stability through immune defense, immune surveillance, and immune homeostasis. During the development and progression of malignant tumors, a dynamic interaction process is established between the tumor and the systemic immune system. Based on this, researchers proposed the hypothesis of “cancer immunoediting”, which indicates three phases of interaction between tumors and the immune system, including “elimination”, “equilibration”, and “escape”. In recent years, with the deepening of research on anti-tumor immunity, significant breakthroughs have been achieved in tumor immunotherapy. Emerging immunotherapeutic strategies, such as monoclonal antibody therapy targeting the PD-1/PD-L1 pathway, have achieved remarkable efficacy in treating malignant tumors such as melanoma, lung cancer, and bladder cancer. These advancements have greatly inspired researchers' enthusiasm for tumor immunology studies. Therefore, restoring the body's normal anti-tumor immunity is considered as a promising curative treatment strategy for malignant tumors.

With the release of numerous clinical trial results for immunotherapy, the variability in therapeutic efficacy of immune checkpoint inhibitors targeting PD-1/PD-L1 has become increasingly evident across different types of cancer and among individuals with the same type of cancer. Even in traditionally considered immune “hot” tumors, such as melanoma, lung cancer, and bladder cancer, a significant proportion of patients remain unresponsive to immune checkpoint inhibitors. In anti-PD-L1 therapy, even when PD-L1 expression is assessed in patients prior to treatment and the sensitivity of PD-L1-positive patients to immunotherapy is analyzed, more than 50% of patients remain resistant to treatment. Moreover, even among patients who initially respond well to immunotherapy, some eventually experience tumor progression during treatment. The former is referred to as “primary immunotherapy resistance”, while the latter is referred to as “secondary immunotherapy resistance”. In the clinical exploration of immunotherapy for traditionally considered immune “cold” tumors, such as breast cancer and pancreatic cancer, researchers first conducted stratified analyses of different molecular subtypes based on tumor mutational burden and lymphocyte infiltration to assess the potential of tumor immunotherapy. For example, in triple-negative and HER2-positive breast cancer, researchers found relatively higher tumor mutational burden and greater lymphocyte infiltration. Furthermore, among patients with the same type of tumor, the tumor mutational burden and the levels of lymphocyte infiltration were associated with favorable prognosis, suggesting that immunotherapy may have certain therapeutic potential even in immune cold tumors. As a result, numerous clinical trials of combination immunotherapy have been approved globally for the treatment of cohorts of patients with immune cold tumors, such as breast cancer. In the monotherapy model using immune checkpoint inhibitors, patients did not show significant survival benefits (phase II KEYNOTE-086 study). In contrast, in trials of combination therapy strategies, the Phase III randomized controlled trial IMpassion130 analyzed the efficacy of combining paclitaxel with a PD-L1 monoclonal antibody compared to paclitaxel alone in breast cancer patients. The interim analysis results showed no significant difference in overall survival between the PD-L1 monoclonal antibody combination therapy group and the paclitaxel monotherapy group. However, stratified analysis revealed that in patients with PD-L1-positive immune cells, patients in the PD-L1 monoclonal antibody combined with paclitaxel therapy group showed better survival benefits. Nevertheless, the overall benefit for patients remains limited.

Apart from immune checkpoint inhibitors, immune cell reinfusion therapy and tumor vaccines are two other significant forms of immunotherapy. However, due to the presence of a complex regulatory network within the tumor microenvironment of solid tumors, immune cells reinfused into the patient or anti-tumor immune cells produced by the body stimulated by tumor vaccines often become rapidly exhausted or die upon reaching the partial site of tumor, which greatly diminishes the anti-tumor efficacy, thereby significantly hindering the practical clinical translation and application.

Currently, there is considerable individual variability in the efficacy of immunotherapy for malignant tumors. The root causes lie, on one hand, in the insufficient understanding of the mechanisms underlying immune escape in malignant tumors in basic research, and on the other hand, in the failure to achieve clinical translation of recently discovered targeted intervention strategies for anti-tumor immune regulatory nodes.

In the “cancer-immunity cycle”, the realization of effective anti-tumor immunity requires seven steps: (1) the release of tumor antigens and their capture by DC cells; (2) the presentation of antigens by DC cells to T cells; (3) the activation of T cells in draining lymph nodes; (4) the migration of activated T cells to the tumor bed; (5) the infiltration of activated T cells into the tumor through the blood vessel; (6) the recognition of tumor cells by activated effector T cells; and (7) the killing of tumor cells by T cells. Any abnormality in any of these steps in the cycle can lead to the occurrence of anti-tumor immune escape. Currently, the PD-1 and PD-L1 antagonists used in clinical practice only target step (7) of this cycle.

Furthermore, in solid tumors, the complex tumor microenvironment serves as a critical protective shield that contributes to tumor immune escape. In addition to tumor cells themselves, the tumor microenvironment includes endothelial cells, fibroblasts, and pericytes in the stroma; immune cells from infiltration like lymphocytes, macrophages, granulocytes, and myeloid-derived suppressor cells; extracellular matrix components; a large amount of cytokines and chemokines; and the like. In recent years, numerous experimental studies on tumors and predictive models of tumor biological behavior have demonstrated that the tumor microenvironment plays a pivotal role in tumorigenesis, progression, distant metastasis, and therapeutic resistance in malignant tumors. With the continuous advancement of research into the tumor microenvironment, researchers have gradually realized that the interactions among various cellular components in the tumor microenvironment are dynamically changing as the tumor progresses or with the intervention of different therapeutic strategies. Moreover, the cellular components in the microenvironment exhibit high heterogeneity, with the same cell type potentially displaying both pro-tumor and anti-tumor phenotypes simultaneously. Therefore, in-depth studies on the dynamic changes in anti-tumor immunity during different stages of tumorigenesis, tumor development, and treatment, particularly the impact of the interactions among various components of the tumor microenvironment on anti-tumor immunity, are of importance. This includes multi-dimensional and multi-faceted analyses of the activation and recruitment of anti-tumor immune cells, as well as the regulatory effects of the tumor microenvironment on the function and survival of these immune cells. By exploring the regulatory networks of anti-tumor immunity in solid tumors and identifying key molecular regulatory nodes within these networks, researchers can provide new therapeutic targets for immunotherapy. This will undoubtedly become a critical direction and goal for future research in tumor immunotherapy.

### SUMMARY

In view of the direction and the target described above, the present disclosure starts from the interaction of the stroma cell components of the tumor microenvironment and the killer lymphocytes mainly executing the anti-tumor immune function, analyzes the key regulation and control nodes of the specific fibroblast subset-mediated killer lymphocyte incapacity in the microenvironment, identifies the new immune checkpoint molecules, and blocks the death of the killer lymphocytes mediated by the fibroblasts by targeting CD158d immune checkpoint molecules or HS3ST3B1-positive cancer-associated fibroblasts, thereby recovering the killing and clearing capacity of the lymphocytes to the tumor cells.

Therefore, in one aspect, the present disclosure provides use of an HS3ST3B1-positive cancer-associated fibroblast for manufacturing a kit for the detection of the death of killer lymphocytes.

In another aspect, the present disclosure provides use of an HS3ST3B1-positive cancer-associated fibroblast for manufacturing a kit for the detection of tumor immune escape.

In another aspect, the present disclosure provides use of HS3ST3B1 as a target for screening or manufacturing a medicament for the inhibition of tumor immune escape.

In another aspect, the present disclosure provides an immune checkpoint molecule CD158d, which mediates tumor fibroblast-mediated death of killer lymphocytes.

In another aspect, the present disclosure provides use of a CD158d molecule as an immune checkpoint, which mediates tumor fibroblast-mediated death of killer lymphocytes.

In another aspect, the present disclosure provides use of a CD158d molecule as a target for screening or manufacturing a medicament for the inhibition of tumor immune escape. In another aspect, the present disclosure provides a neutralizing antibody binding to a CD158d molecule, which inhibits tumor immune escape by neutralizing CD158d.

In a preferred embodiment of the present disclosure, the neutralizing antibody is a monoclonal antibody or a polyclonal antibody.

In another aspect, the present disclosure provides use of the neutralizing antibody described herein as an immune checkpoint inhibitor.

In another aspect, the present disclosure provides use of the neutralizing antibody for manufacturing a medicament for the inhibition of tumor immune escape.

### Beneficial Effects

Compared with the prior art, the present disclosure discovers that the HS3ST3B1-positive cancer-associated fibroblasts can mediate the death of killer lymphocytes. Based on this, the present disclosure further finds a novel immune checkpoint molecule CD158d, which can mediate tumor fibroblast-mediated death of killer lymphocytes. Based on the findings described above, the present disclosure develops use of a CD158d molecule and HS3ST3B1 as a target for screening or manufacturing a medicament for the inhibition of tumor immune escape. Aiming at the fact that the CD158d molecules can mediate tumor fibroblast-mediated death of killer lymphocytes, the present disclosure obtains a neutralizing antibody capable of neutralizing the CD158d molecules, and after the antibody neutralizes the CD158d molecules, the killing effect of CTLs on tumors can be remarkably restored. Therefore, the present disclosure provides use of a CD158d neutralizing antibody for manufacturing a medicament for the inhibition of tumor immune escape.

### BRIEF DESCRIPTION OF THE DRAWINGS

FIG. 1 illustrates the trend showing that silencing HS3ST3B1 in HS3ST3B1-positive cancer-associated fibroblasts inhibits the induced death of cytotoxic lymphocytes.

A. Representative flow cytometry plots showing AnnexinV fluorescence antibody and PI staining in untreated CTLs (CTRL group) and CTLs in groups co-cultured with different fibroblasts.

The red numbers indicate the proportion of AnnexinV-positive cells (second and fourth quadrants), i.e., the proportion of dead cells.

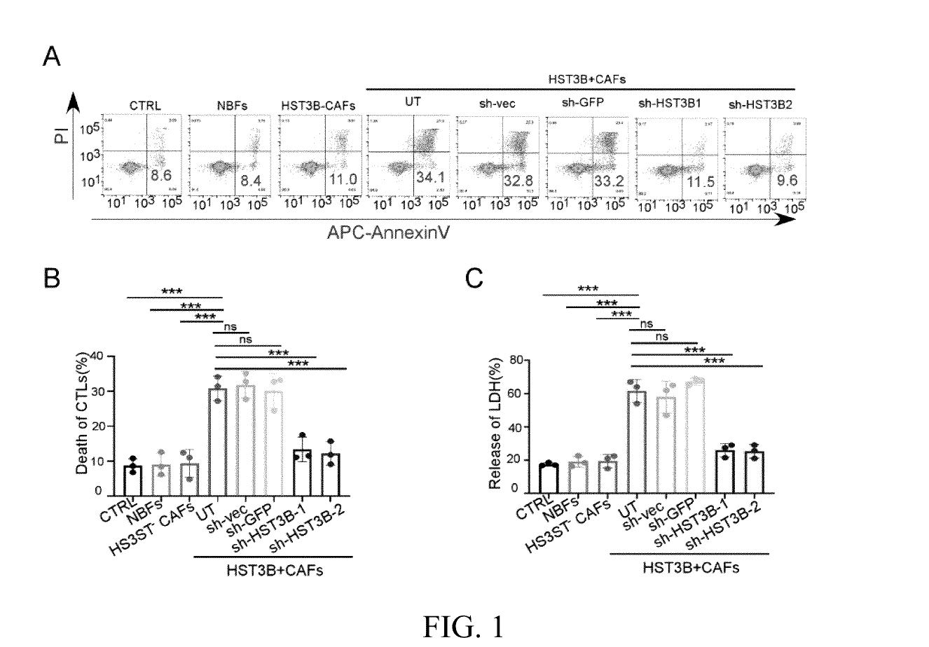

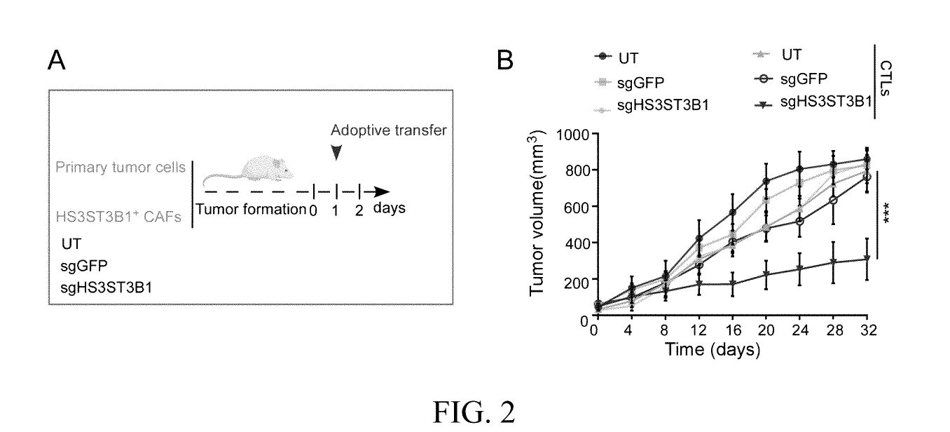

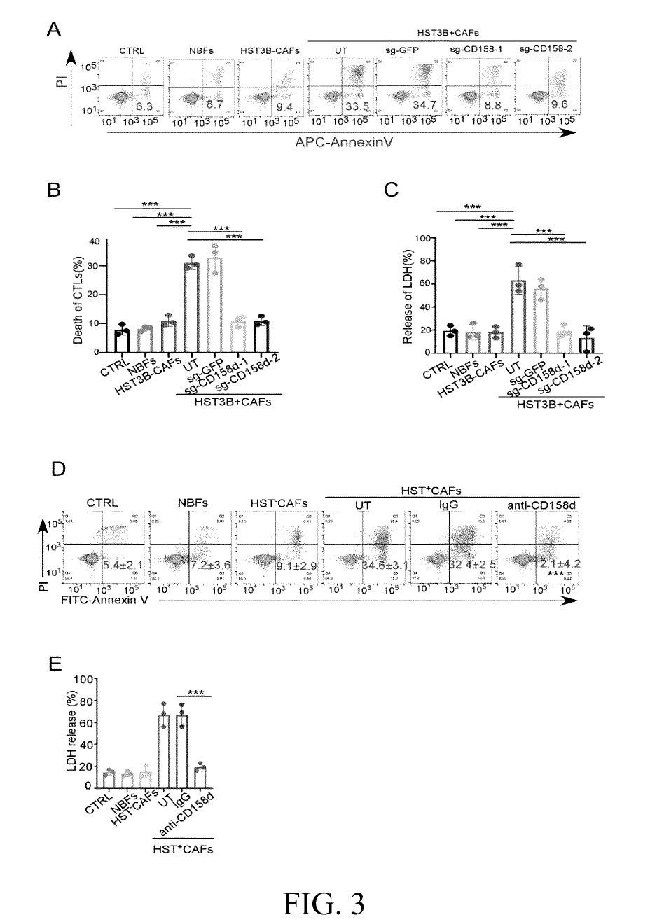

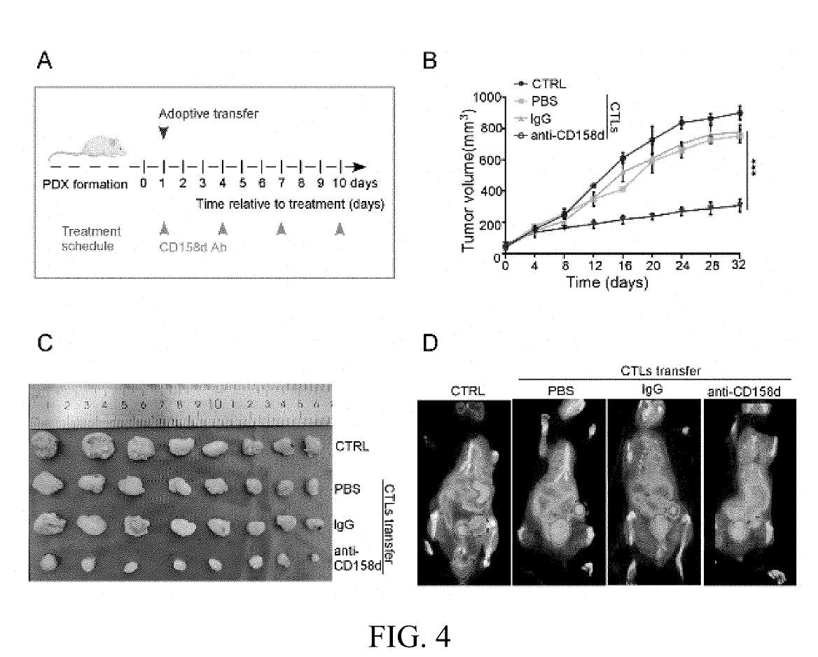

B. Quantification of FIG. 1A (n=3).

C. Measurement of lactate dehydrogenase (LDH) release in the culture system of untreated CTLs (CTRL group) and culture system of CTLs in groups co-cultured with different fibroblasts. The proportion of LDH release represents the proportion of dead cells (n=3).

FIG. 2 illustrates the enhancement of in vivo tumor immunity after silencing HS3ST3B1 in HS3ST3B1-positive cancer-associated fibroblasts.

A. Schematic representation of xenograft tumors in mice co-injected with primary tumor cells and differently treated HS3ST3B1⁺ CAFs.

B. Growth of various xenograft tumors in mice co-injected with primary tumor cells and differently treated HS3ST3B1⁺ CAFs, without treatment or with reinfusion of CTLs (n=8).

FIG. 3 illustrates the schematic representation of the termination of the death trend mediated by HS3ST3B1⁺ CAFs after CD158d is knocked out in CTLs or neutralized using a neutralizing antibody.

A. Representative flow cytometry plots showing AnnexinV fluorescence antibody and PI staining in CTLs cultured alone or co-cultured with different fibroblasts in various groups (UT, sgGFP, and CD158d knockout groups).

The red numbers indicate the proportion of AnnexinV-positive cells.

B. Quantification of FIG. 3A (n=3).

C. Detection of LDH release (n=3) after CTLs cultured alone or co-cultured with different fibroblasts in various groups (UT, sgGFP, and CD158d knockout groups).

D. Flow cytometry analysis showing AnnexinV fluorescence antibody and PI staining (n=3) after CTLs cultured alone or co-cultured with different fibroblasts in various groups (UT, IgG, and CD158d neutralizing antibody groups).

E. Detection of LDH release (n=3) after CTLs cultured alone or co-cultured with different fibroblasts in various groups (UT, IgG, and CD158d neutralizing antibody groups).

FIG. 4 illustrates the promotion of tumor immunity by targeting CD158d in vivo using neutralizing antibodies.

A. Schematic representation of targeted CD158d in combination with adoptive immunization on PDX mice.

B. Tumor growth rates of PDX tumors in mice from the group without CTL infusion and groups with CTL reinfusion subjected to different treatments (PBS, IgG, and CD158d neutralizing antibody treatments).

C. Sizes of all PDX tumors in mice from the group without CTL infusion and groups with CTL reinfusion subjected to different treatments (PBS, IgG, and CD158d neutralizing antibody treatments).

D. Representative PET/CT images showing the sizes of PDX tumors in mice from the group without CTL infusion and groups with CTL reinfusion subjected to different treatments (PBS, IgG, and CD158d neutralizing antibody treatments).

### DETAILED DESCRIPTION

For better illustrating the objectives and advantages of the present method, the detailed description of the present disclosure will be further described in detail with reference to the accompanying drawings and specific examples.

### Example 1: HS3ST3B1⁺ CAFs Induce Death of CTLs by Expressing HS3S3ST3B1

Cancer-associated fibroblasts (CAFs) and normal breast fibroblasts (NBFs) were isolated from primary tissues of breast cancer patients or breast tissues from patients undergoing breast reduction surgery.

CAFs were divided into HS3ST3B1-negative and HS3ST3B1-positive groups using flow cytometric sorting.

When the fibroblasts described above were co-cultured with cytotoxic T lymphocytes (CTLs), it was observed that in comparison to normal breast fibroblasts (NBFs) and HS3ST3B1-negative cancer-associated fibroblasts (HS3ST3B1⁻ CAFs), HS3ST3B1-positive cancer-associated fibroblasts (HS3ST3B1⁺ CAFs) significantly induced the death of CTLs, see A, B, and C of FIG. 1.

When fibroblasts were infected with lentiviruses encoding shRNAs to silence HS3S3ST3B1 expression, the death of CTLs induced by HS3ST3B1⁺ CAFs was inhibited, which participated in A, B, and C of FIG. 1.

The results described above indicate that HS3ST3B1⁺ CAFs induce the death of CTLs by expressing HS3S3ST3B1.

In this example, the sorting of HS3ST3B1-positive cancer-associated fibroblasts from tumors mainly comprises the following steps:
1. Approximately 1 cubic centimeter of fresh breast cancer tissue from patients was collected.
2. The tissue obtained in step 1 was minced into fragments of about 1 cubic millimeter using sterile scissors, and 10 mL of culture medium containing 1.5 mg/mL type I collagenase and 1.5 mg/mL type III collagenase was added.
3. The mixture in step 2 was incubated in a 37° C. incubator for 2 h.
4. The tissue culture solution in step 3 was filtered to obtain a primary cell suspension.
5. The cell suspension in step 4 was centrifuged. After the cells were resuspended, the cell suspension was incubated with an anti-PDGFRa antibody for flow cytometry and an anti-HS3ST3B1 primary antibody for 30 min. After centrifugation to remove the supernatant, the cells were resuspended, and the cell suspension was incubated with a fluorescent secondary antibody for 45 min. Finally, the cells were centrifuged, the supernatant was discarded, and the cells were resuspended.
6. A flow cell sorter was used to sort the cell suspension in step 5, thereby obtaining HS3ST3B1-positive cancer-associated fibroblasts.

In this example, the construction of the fibroblast and lymphocyte co-culture model mainly comprises the following steps:
1. HS3ST3B1-positive cancer-associated fibroblasts and killer lymphocytes were co-cultured. The fibroblasts were seeded in a transwell lower chamber, while the cytotoxic lymphocytes were placed in the upper chamber.
2. The cell death of co-cultured cytotoxic lymphocytes was measured (LDH release assay, flow cytometry).

### Example 2: HS3ST3B1⁺ CAFs Promote Tumor Cell Immune Escape Via HS3ST3B1 In Vivo

As shown in FIG. 2A, primary tumor cells were isolated from breast cancer tissues, and the tumor cells were co-injected with differently treated HS3ST3B1⁺ CAFs into the fourth pair of mammary fat pads of immunodeficient mice. Also, monocytes were isolated from the peripheral blood of patients and induced to differentiate into dendritic cells, which were then loaded with tumor antigens. The dendritic cells loaded with tumor antigens were subsequently used to treat the CD8 T lymphocytes isolated from peripheral blood, thereby constructing tumor-specific CTLs in vitro, and tail vein reinfusion was performed when the mouse tumor reached a diameter of 2 mm.

The present disclosure revealed that xenograft tumors formed by tumor cells mixed with HS3ST3B1⁺ CAFs resisted the immune killing effects of adoptively reinfused CTLs. This was evidenced by the significant growth of the xenograft tumors even after CTL adoptive reinfusion, see FIG. 2B. However, when the expression of HS3ST3B1 in this population of CAFs was silenced, the adoptive immune function was significantly restored. These findings demonstrate that HS3ST3B1⁺ CAFs promote tumor cell immune escape via HS3ST3B1 in vivo.

In this embodiment, the in vivo validation model of immune escape mediated by HS3ST3B1-positive fibroblasts mainly comprises the following steps:
1. HS3ST3B1-positive cancer-associated fibroblasts were sorted, and the HS3ST3B1 gene in the fibroblasts was knocked out using the crispr-cas9 technology.
2. Primary tumor cells were sorted from patient tumor tissues.
3. Fibroblasts in knockout HS3ST3B1 group and non-knockout group were separately mixed with the primary tumor cells and implanted into the fat pads of immunodeficient mice.
4. Primary tumor cell-specific cytotoxic T lymphocytes were constructed in vitro:
(1) Primary tumor cells were expanded and collected as cell pellets, which were subjected to five freeze-thaw cycles;
(2) The supernatant (tumor cell lysate) was collected after centrifugation at 14000 rpm;
(3) Monocytes were isolated from the peripheral blood of the patient and induced into DC cells by adding IL4 and GMSCF cytokines;
(4) Tumor lysates were added to the DC cell culture medium;
(5) T lymphocytes from the same source were added and co-cultured for five days, and CD8-positive CTL cells were sorted.
5. CTL cells were reinfused into the immunodeficient mouse models constructed in step 3 via tail vein injection.
6. Tumor progression in the mice was observed.

### Example 3: HS3ST3B1⁺ CAFs Mediate Death of CTLs Via CD158d Molecule on CTLs

When the expression of CD158d in CTLs was silenced using CRISPR-CAS9, the HS3ST3B1+ CAF-induced the death of CTLs tendency was inhibited, see A, B, and C of FIG. 3.

Furthermore, a CD158d neutralizing antibody was constructed in vitro and added to the co-culture system of the CAFs and CTLs. It was observed that the addition of the CD158d neutralizing antibody can significantly inhibit HS3ST3B1⁺ CAF-induced death of CTLs, see E and F of FIG. 3. This indicates that HS3ST3B1⁺ CAFs mediated the death of CTLs via the CD158d molecule on CTLs, in which CD158d was a checkpoint molecule mediating immunosuppression.

In this example, the neutralizing antibody construction experiment mainly comprises the following steps:
1. The protein sequence of in vitro recombinant human CD158d full-length protein (Ag) is as shown in Sequence 1.
2. Mouse immunization:
(1) Primary immunization: 50 μg of Ag per mouse was administered with Freund's complete adjuvant via subcutaneous multi-point injection, and the injection was typically 1.5 mL at an interval of 3 weeks;
(2) Second immunization: the same dose and route as above were used with Freund's incomplete adjuvant at an interval of 3 weeks;
(3) Third immunization: the same dose as above was used without an adjuvant via intraperitoneal injection, and blood was collected 7 days later to measure the titer and detect the immunization efficacy at an interval of 3 weeks;
(4) Booster immunization: the dose was 50 μg via intraperitoneal injection;
(5) Spleen was taken for fusion after 3 days.
3. Cell fusion:
(1) Preparation of feeder cells:
preparation of mouse peritoneal macrophages:
\textcircled{1} BALB/c mice at 6-10 weeks of age were used;
\textcircled{2} cervical dislocation was performed to euthanize the mouse, followed by immersion in 75% ethanol for 3 min for disinfection, the skin was cut open with sterile scissors to expose the peritoneum, 6 mL of culture medium was injected using a pipette and washed repeatedly, and the lavage fluid was aspirated;
\textcircled{3} the lavage fluid was placed in a 10 mL centrifuge tube and centrifuged at 1200 rpm for 5 min;
\textcircled{4} the cells were resuspended in culture medium containing 20% fetal bovine serum, the cell density was adjusted to 1×10⁵/mL, and 100 μL was added per well to a 96-well plate; the plate was incubated in an incubator at 37° C.;
(2) Preparation of myeloma cells:
\textcircled{1} myeloma cells were subjected to an expansion culture 48-36 h before fusion;
\textcircled{2} on the day of fusion, the cells were gently dislodged from the wall of the bottle using a bent-tip pipette and collected in a 50 mL centrifuge tube or fusion tube;
\textcircled{3} the cells were centrifuged at 1000 r/min for 5-10 min, and the supernatant was discarded;
\textcircled{4} 30 mL of incomplete culture medium was added, the cells were centrifuged and washed once, and the cells were then resuspended in 10 mL of incomplete culture medium and mixed uniformly;
\textcircled{5} a myeloma cell suspension was taken, and viable cells were counted using 0.4% trypan blue staining for later use.
(3) Preparation of splenocytes

Blood was collected from immunized BALB/c mice by enucleation of eyeball, and the serum was separated for use as a positive control in antibody detection. The mice were euthanized by cervical dislocation and immersed in 75% ethanol for 5 min. The mice were fixed on a culture dish, the skin on the left side of the abdomen was lifted, and the spleen was exposed. A pair of ophthalmological scissors and forceps was used. Peritoneum was cut in an ultra clean bench by aseptic operation, and the spleen was carefully taken out and placed on a dish containing 10 mL of incomplete culture medium. The mixture was gently washed, and surrounding connective tissues were carefully removed. The mixture was placed on a stainless-steel screen on a dish, ground into a cell suspension with a syringe needle core, and counted, so that the spleen cells entered into the incomplete culture medium on the dish. The mixture was pipetted several times to obtain a single cell suspension. Typically 1×10⁸ to 2.5× 10⁸ spleen cells per mouse.
(4) Cell fusion
\textcircled{1} 1×10⁸ spleen cells and 1×10⁷ myeloma cells SP2/0 were mixed in a 50 mL fusion tube, an incomplete culture medium was supplemented to 30 mL, and the mixture was mixed uniformly;
\textcircled{2} the mixture was centrifuged at 1000 rpm for 5-10 min, and the supernatant was removed to the greatest extent;
\textcircled{3} the fusion tube was tapped on the bottom on the palm to loosen the cell pellet uniformly;
\textcircled{4} 1 mL of pre-warmed 50% PEG was added using a 1 mL pipette within 30 s while gently stirring;
\textcircled{5} the mixture was pipetted into a pipette and left to stand for 1 min;
\textcircled{6} a pre-warmed incomplete culture medium was added, and the PEG reaction was terminated with 1 mL, 2 mL, 3 mL, 4 mL, 5 mL, and 10 mL continuously added at 2-minute intervals;
\textcircled{7} the mixture was centrifuged at 800 rpm for 5 min, and the supernatant was discarded;
\textcircled{8} 5 mL of a complete culture medium was added, and the cell pellet was gently pipetted to suspend and mix the cells uniformly; then a complete culture medium was supplemented to 40-50 mL, and the mixture was added to 96-well cell culture plates at 100 μL per well; then the plates were incubated in an incubator at 37° C. with 5% CO₂;
\textcircled{9} after 6 h, a selective culture medium was supplemented at 50 μL per well, and a half medium exchange was performed with a selective culture medium after 3 days;
\textcircled{10} the growth of the hybridoma cells was often monitored, and when they covered 1/10 or more of the bottom area, the supernatant was pipetted for antibody detection.
(5) Selection of hybridoma cells:
\textcircled{1} the antigen was diluted to 10 μg/mL with a coating solution;
\textcircled{2} 100 μL was added per well to a microplate and incubated at 4° C. overnight or adsorbed at 37° C. for 2 h;
\textcircled{3} the liquid in the wells was discarded, and the wells were washed 3 times with washings, 3 min each time, and tap dried;
\textcircled{4} 100 μL of a blocking solution was added per well, followed by blocking at 37° C. for 1 h;
\textcircled{5} the wells were washed 3 times with washings;
\textcircled{6} 100 μL of the hybridoma cell culture supernatant to be detected was added per well; positive, negative, and blank controls were included; the plate was incubated at 37° C. for 1 h, washed, and tap dried;
\textcircled{7} 100 μL of an enzyme-labeled secondary antibody was added per well, and the plate was incubated at 37° C. for 1 h, washed, and tap dried;
\textcircled{8} a substrate solution was added, 100 μL of a freshly prepared substrate use solution was added per well, and the plate was incubated at 37° C. for 20 min;
\textcircled{9} the reaction was stopped with 2 mol/L H₂SO₄, and the OD value was read using an enzyme-linked immunosorbent assay reader;
\textcircled{10} result determination: P/N≥2.1 or P≥N1+3 SD was considered as positive; if the negative control wells were colorless or nearly colorless and the positive control wells showed clear coloration, the results can be visually judged.
(6) Cloning of hybridoma cells (limiting dilution method)
\textcircled{1} mouse spleen cells were prepared as feeder cells;
\textcircled{2} the hybridoma cell suspension to be cloned was prepared and diluted in HT culture medium containing 20% serum to 3 different concentrations of 5, 10, and 20 cells per mL;
\textcircled{3} peritoneal macrophages were added to the hybridoma cell suspension described above at a ratio of 5×10⁴ to 1× 10⁵ cells per mL;
\textcircled{4} each kind of hybridoma cells was added to a 96-well plate at 100 μL per well;
\textcircled{5} the cells were incubated at 37° C. with 5% CO₂ for 6 days; when clones visible to the naked eye appeared, antibodies can be detected under an inverted microscope; the wells with a single clone were marked, and the supernatant was collected and subjected to antibody detection;
\textcircled{6} the cells that were in the positive wells in the antibody detection were expanded and cryopreserved.
4. Identification of Ig class and subclass of monoclonal antibodies
(1) The microplates were coated with 10 μg/mL antigen (50 μL per well) and incubated overnight at 4° C.;
(2) After washing, 100 μL of monoclonal antibody samples was added per well, and the plates were incubated at 37° C. for 1 h. Positive and negative control wells were included;
(3) After washing, 100 μL of HRP-labeled anti-mouse Ig and subclass Ig antibody reagent was added per well and subjected to color development at 37° C. for 20 min in the dark. The reaction was stopped with 2 mol/L H₂SO₄, and the antibody subclass was determined based on the color.
5. Production and purification of monoclonal antibodies
(1) In vivo production of monoclonal antibodies in animals
\textcircled{1} pristane or liquid paraffin were intraperitoneally injected into adult BALB/c mice at 0.3-0.5 mL per mouse;
\textcircled{2} after 7-10 days, hybridoma cells diluted with PBS or serum-free culture medium were injected intraperitoneally at 5×10⁵/0.2 mL per mouse;
\textcircled{3} ascites production was monitored daily after 5 days; when the abdomen was visibly distended and the skin felt tense when touched by hands, ascites can be collected; typically, 3 mL of ascites can be collected per mouse;
\textcircled{4} the ascites were centrifuged at 2000 rpm for 5 min to remove cellular components and other precipitates; the supernatant was collected for antibody titer determination, aliquoted, and cryopreserved at −70° C. for later use.
(2) Purification of monoclonal antibodies (octanoic acid-ammonium sulfate precipitation method)
\textcircled{1} the ascites were centrifuged at 12000 rpm for 15 min at 4° C. to remove impurities;
\textcircled{2} 2 parts of 0.06 mol/L acetic acid buffer (pH 5.0) were added to 1 part of the ascites, and octanoic acid was added dropwise at room temperature while stirring at a ratio of 33 μL of octanoic acid per mL of the diluted ascites; the mixture was mixed at room temperature for 30 min;
\textcircled{3} the mixture was left to stand at 4° C. for 2 h, taken out, and then centrifuged at 12000 g for 30 min, and the pellet was discarded;
\textcircled{4} the supernatant was filtered through a nylon mesh and dialyzed in 50 times volume of 0.01 M pH 7.4 PBS at 4° C. for 6 h;
\textcircled{5} an equal volume of a saturated ammonium sulfate solution was added to the dialyzed supernatant;
\textcircled{6} the mixture was left to stand at 4° C. for at least 1 h and centrifuged at 10000 g for 30 min, and the supernatant was discarded;
\textcircled{7} the pellet was dissolved in an appropriate amount of PBS (containing 137 mmol/L NaCl, 2.6 mol/L KCl, and 0.2 mmol/L EDTA), and dialyzed overnight in 50-100 times volume of PBS;
\textcircled{8} a small amount of the dialyzed sample was taken, diluted properly, and detected for the protein concentration using an ultraviolet spectrophotometer, and the antibody purity was detected using SDS-PAGE and WB.

In this example, the neutralizing antibody experiment in in vitro models mainly comprises the following steps:
1. A constructed neutralizing antibody was added to a co-culture model of fibroblasts and cytotoxic lymphocytes.
2. The death of cytotoxic lymphocytes was detected.

### Example 4: In Vivo Validation of HS3ST3B1⁺ CAFs Mediating Immunosuppression

To further validate in vivo that HS3ST3B1⁺ CAFs can mediate immunosuppression and that CD158d is an immunosuppression-related checkpoint molecule, the present disclosure collected breast cancer tissues with a high proportion of HS3ST3B1⁺ CAFs from breast cancer patients and constructed PDX model in immunodeficient NOD/SCID mice. The PDX model refers to an in vivo research model in which a xenograft is constructed in immunodeficient mice using tumor tissues derived from patients.

The present disclosure also collected paired peripheral blood samples from breast cancer patients. CD8 T lymphocytes and DCs were isolated in vitro. Tumor tissue lysates were used to activate the DCs, and the DC cells were co-cultured with CD8T lymphocytes from the same source to induce tumor-specific CTLs. As shown in FIG. 4A, after PDX tumor formation, tail vein reinfusion of CTLs was performed, and CD158d neutralizing antibodies were intraperitoneally injected into a portion of mice every 4 days.

The results show that PDX constructed from breast cancer tissues with a high proportion of HS3ST3B1⁺ CAFs can resist the killing of tumor-specific CTLs, see B, C, and D of FIG. 4; after CD158d was neutralized with the neutralizing antibody, the killing effect of CTLs on PDXs was significantly restored, see B, C, and D of FIG. 4). This suggests that enrichment of HS3ST3B1+ CAFs leads to immunosuppression, and targeting CD158d can significantly restore tumor immunity.

In this example, the treatment of the mice with the neutralizing antibody in an in vivo experimental model mainly comprises the following steps:
1. Construction of immunodeficient mouse PDX model
(1) Fresh tumor tissues were cut into 1 cubic millimeter tissue particles with sterile scissors;
(2) The tissue particles were implanted into the mouse fat pad;
(3) The tissues after the tumor formation were then passaged.
2. In vitro construction of tumor-specific cytotoxic T lymphocytes
(1) Tumor tissues from the same patient were ground and subjected to five freeze-thaw cycles;
(2) The supernatant was collected after centrifugation at 14000 rpm;
(3) Monocytes were isolated from the peripheral blood of the patient and induced into DC cells by adding IL4 and GMSCF cytokines;
(4) Tumor lysates were added to the DC cells;
(5) T lymphocytes from the same source were added and co-cultured for five days, and CD8-positive CTL cells were sorted.
3. Adoptive immunization of PDX model, and injection of CTL cell suspension via tail vein.
4. Intraperitoneal injection of constructed neutralizing antibodies every 3 days and tumor size measurement.

The foregoing is the preferred example of the present disclosure, and the present disclosure is not intended to be limited to the content disclosed in the example and the accompanying drawings. All equivalents and modifications obtained without departing from the spirit disclosed in the present disclosure shall fall within the protection scope of the present disclosure.

1. A method of manufacturing a kit for the detection of the death of killer lymphocytes or tumor immune escape, comprising using an HS3ST3B1-positive cancer-associated fibroblast.

2. (canceled)

3. A method of screening or manufacturing a medicament for the inhibition of tumor immune escape using the kit of claim 1, comprising using HS3ST3B1 as a target.

4. An immune checkpoint molecule CD158d, which mediating tumor fibroblast-mediated death of killer lymphocytes.

5. A method of using a CD158d molecule as an immune checkpoint of claim 4, wherein the molecule mediates tumor fibroblast-mediated death of killer lymphocytes.

6. A method of using a CD158d molecule of claim 4 as a target for screening or manufacturing a medicament for the inhibition of tumor immune escape.

7. A neutralizing antibody binding to a CD158d molecule of claim 4, wherein the antibody inhibiting tumor immune escape by neutralizing CD158d.

8. The neutralizing antibody according to claim 7, wherein the neutralizing antibody is a monoclonal antibody or a polyclonal antibody.

9. A method of using the neutralizing antibody as claimed in claim 7 as an immune checkpoint inhibitor.

10. A method of inhibiting tumor immune escape, comprising using the neutralizing antibody as claimed in claim 7.
